(19) United States
(12) Patent Application Publication
(10) Pub. No.: US 2026/0026908 A1
(43) Pub. Date: Jan. 29, 2026

(54) CABINET FOR STORING SURGICAL INSTRUMENTS AND METHOD FOR OPERATING A CABINET FOR STORING SURGICAL INSTRUMENTS

(71) Applicant: OLYMPUS Winter & Ibe GmbH, Hamburg (DE)

(72) Inventor: Oliver HOPF, Pinneber (DE)

(73) Assignee: OLYMPUS Winter & Ibe GmbH, Hamburg (DE)

(21) Appl. No.: 19/065,829

(22) Filed: Feb. 27, 2025

(30) Foreign Application Priority Data
Jul. 29, 2024 (DE) 10 2024 121 513.5

(51) Int. Cl. A61B 50/30

(52) U.S. Cl. CPC A61B 50/30; A61B 2050/3004

(57) ABSTRACT

A cabinet for storing surgical instruments the cabinet including: a casing, wherein the casing encloses an interior space with at least one mounting device configured to hold a surgical instrument; a ventilation unit configured to supply drying gas to the interior space; and a cooling unit and a drying unit connected to the ventilation unit, wherein the drying unit is configured to reduce the humidity of the drying gas, and the cooling unit is configured to cool down the drying gas before it enters the interior space to a temperature below 15° C.

### CROSS-REFERENCE TO RELATED APPLICATION

The present application is based upon and claims the benefit of priority from DE 10 2024 121 513.5 filed on Jul. 29, 2024, the entire contents of which is incorporated herein by reference.

### BACKGROUND

### Field

The present disclosure relates to a cabinet for storing surgical instruments. The present disclosure further relates to a method for operating a cabinet for storing surgical instruments.

### Prior Art

Surgical instruments, for example endoscopes, have to be cleaned and disinfected after use. After cleaning, it is necessary to dry the surgical instruments to remove any traces of the cleaning fluid, before they can be used again. For this drying process, drying cabinets are often used, in which the surgical instruments are arranged after cleaning.

However, sometimes the surgical instruments will stay in the drying cabinet for several days or weeks before they are required again. If the surgical instruments remain in the drying cabinet for such a long period of time, there is a risk that they are contaminated again through germs transmitted through ambient air or by touch. Such germs, which might get into contact with a surgical endoscope after disinfection, may include for example Escherichia coli, Pseudomonas aeruginosa, Staphylococcus aureus, streptococci, Candida albicans or Aspergillus fumigatus. All these types of germs are potentially harmful during an operation and may contaminate the surgical instruments while they are stored inside the drying cabinet.

### SUMMARY

An object is to better protect surgical instruments from recontamination by germs after cleaning and disinfection during the drying or storage phase.

Such object can be solved by a cabinet for storing surgical instruments, such as endoscopes, wherein the cabinet comprises a ventilation unit and a casing, wherein the casing encloses an interior space with at least one mounting device, wherein the mounting device is configured to hold a surgical instrument, wherein the ventilation unit is configured to supply drying gas to the interior space, wherein the cabinet further comprises a cooling unit and a drying unit connected to the ventilation unit, the drying unit is configured to reduce the humidity of the drying gas, and the cooling unit is configured to cool down the drying gas before it enters the interior space to a temperature below 15° C.

By cooling down the drying gas before it enters the interior space, the probability of recontamination with germs can be sharply reduced. The reason for this is that the types of germs, which are typically responsible for the recontamination, all thrive poorly at low temperatures. Thus, by cooling down the drying gas, the environmental conditions for the germs are strongly impaired.

The drying unit can improve water absorption and thus reduce the humidity of the drying gas in order to improve the drying capacity of the cabinet. The drying unit may be a passive drying unit. For example, the drying unit may comprise a silicate mineral in order to passively dry the drying gas. The drying unit may be an active drying unit. For example, the drying unit may comprise an air drying configured to actively dry the drying gas. The cooling unit and the drying unit may be formed as a single cooling and drying unit. The drying gas may be air, a noble gas or a mixture of different gases. The cabinet may comprise a control system for controlling the ventilation unit and the cooling unit and the drying unit.

The cooling unit can be configured to cool down the drying gas before it enters the interior space to a temperature below 10° C., such as to a temperature of 6° C. to 8° C., for example, a temperature of about 7° C., as it can greatly inhibit the recontamination with germs and offers good storage conditions for the surgical instruments.

The interior space can be thermally insulated and airtight against the exterior of the cabinet. The thermal insulation and the airtight configuration can make the operation of the cooling unit much more efficient, so that it takes less energy to cool down the drying gas and keep it at low temperatures. In addition, the recontamination of surgical instruments by germs from the exterior can be prevented by the airtight configuration.

According to an embodiment, the cabinet can comprise at least one door, wherein the door is sealed against the casing with an airtight sealing in a closed state. The airtight sealing can prevent ambient air from entering the interior space through small gaps between the door and the casing. The door may have two door wings.

The cabinet can comprise a closed-off circulation system for the drying gas, wherein the closed off circulation system can comprises the interior space, the ventilation unit and the cooling unit. In other words, the cabinet can provide a closed loop for the drying gas inside the cabinet. For example, there is no air intake for ambient air and no intake for gas from outside the cabinet. Instead, the drying gas can be continuously circulated through the interior space, the ventilation unit and the cooling unit in order to keep the temperature of the drying gas inside the interior space cool. This not only can prevent the recontamination via germs from the outside but can also save energy during the operation of the cabinet.

The ventilation unit can be configured to circulate the drying gas between the interior space and the cooling unit and the drying unit. The ventilation unit may comprise one or more ventilation devices to circulate the drying gas. The ventilation devices may comprise at least one ventilator or fan.

According to an embodiment, the drying unit and/or the ventilation unit and/or the cooling unit can comprise a filter, wherein the filter is configured to continuously filter the drying gas. By continuously filtering the drying gas, any remaining germs and other contaminations are removed from the drying gas during the operation of the cabinet. Thus, the filter can prevent recontamination of the surgical instruments. The filter can be a sterile filter. The filter can be configured to filter the drying gas coming from the interior space, before the filtered gas is guided back towards the interior space or towards the cooling unit.

The ventilation unit can be configured to supply the drying gas as a drying gas stream to at least one interior channel of a surgical instrument held in the mounting device. By supplying the drying gas stream to the interior channels of the surgical instrument, these interior channels can also be provided with cooled-down drying gas. In this way, the cabinet can prevent recontamination of the outer surface and the interior channels of the surgical instrument.

According to an embodiment, the cooling unit can be configured to cool down the drying gas during a storage phase, which can happen after a drying phase. The drying phase is a phase during the operation of the cabinet immediately after the surgical instrument is placed in the interior space. Typically, the surgical instrument is still wet from the cleaning process and needs to be dried. Thus, during the drying phase, the drying gas can be utilized to remove any remaining cleaning fluid. During the storage phase, which can happen after the drying phase, the drying of the surgical instrument has already finished and the surgical instrument is stored inside the cabinet until its next use. The drying gas can be cooled down during the storage phase as a recontamination typically happens during this phase, as the storage phase may last a lot longer than the drying phase. The cooling unit and/or the control system can be configured to operate in a way, that a temperature of the drying gas is higher during the drying phase than during the storage phase because during the drying phase a higher temperature can accelerate the removal of the remaining cleaning fluid. An air pressure of the drying gas can be higher during the drying phase than during the storage phase.

According to an embodiment, the cabinet can comprises multiple mounting devices. The ventilation unit can be configured to provide each mounting unit separately with the drying gas. The cabinet may comprise a separate vent or nozzle for each mounting device. The vents or nozzles can be arranged in proximity to the associated mounting devices. The vents or nozzles can be configured to create a separate drying gas stream for each surgical instrument arranged in the mounting devices. In this way, the surgical instruments may be supplied with drying gas streams with different properties. For example, a surgical instrument that is still wet may be supplied with a stronger drying gas stream, whereas a surgical instrument in the storage phase may be supplied with a weaker drying gas stream.

Such object can be further solved by a method for operating a cabinet for storing surgical instruments, such as endoscopes, wherein the cabinet comprises a ventilation unit and a casing, the casing encloses an interior space with at least one mounting device, the mounting device holds a surgical instrument, the ventilation unit supplies drying gas to the interior space, the cabinet further comprises a cooling unit connected to the ventilation unit, and the cooling unit cools down the drying gas before it enters the interior space to a temperature below 15° C.

The method can embody the same advantages and characteristics as the cabinet described above.

The cooling unit can cool down the drying gas before it enters the interior space to a temperature below 10° C., such as to a temperature of 6° C. to 8° C.

The cabinet can circulate the drying gas between the interior space and the cooling unit.

According to an embodiment, the drying unit and/or the ventilation unit and/or the cooling unit can comprise a filter, wherein the filter can continuously filter the drying gas.

The ventilation unit can supply the drying gas as a drying gas stream to at least one interior channel of the surgical instrument held in the mounting device.

The cooling unit can cool down the drying gas during a storage phase, which can happen after a drying phase.

### BRIEF DESCRIPTION OF THE DRAWINGS

Further features will become apparent from the description of the embodiments together with the claims and the included drawings. Embodiments can fulfill individual characteristics or a combination of several characteristics.

The embodiments are described below, without restricting the general intent of the invention, based on exemplary embodiments, wherein reference is made expressly to the drawings with regard to the disclosure of all details that are not explained in greater detail in the text. In the drawings:

FIG. 1 illustrates a schematic simplified perspective representation of a cabinet for storing surgical instruments,

FIG. 2 illustrates a schematic simplified close-up of the cabinet and several surgical instruments stored within, and

FIG. 3 illustrates a schematic simplified cross-section of an embodiment of the cabinet for storing surgical instruments.

In the drawings, the same or similar types of elements or respectively corresponding parts are provided with the same reference numbers in order to prevent the item from needing to be reintroduced.

### DETAILED DESCRIPTION

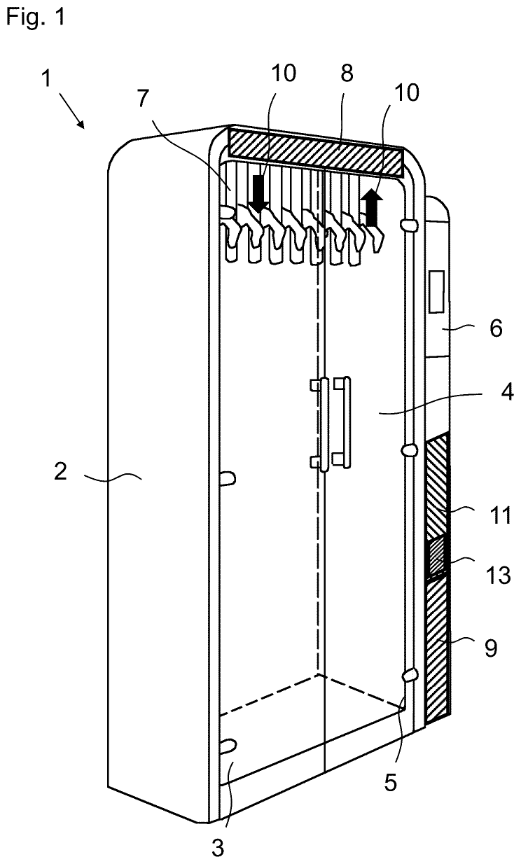

FIG. 1 shows a schematic, simplified perspective drawing of a cabinet 1 for drying and storing surgical instruments. The cabinet 1 comprises a casing 2 and a door 3 with wings (i.e., two doors oppositely hinged). The casing 2 and the door 3 enclose an interior space 4. The interior space 4 is thermally insulated and airtight against the outside of cabinet 1. This is partly achieved by an airtight sealing 5 between the door 3 and the casing 2.

Inside the interior space 4, there are a number of mounting devices 7, which are configured to hold a surgical instrument each, for example an endoscope. The cabinet further comprises a ventilation unit 8, which is configured to supply drying gas 10 to the interior space 4 and circulate the drying gas 10 between interior space 4 and a cooling unit 9. The cooling unit 9 is configured to cool down the drying gas 10 to a temperature of about 7° C. Such a temperature prevents a recontamination of the surgical instrument stored inside the interior space 4. The drying gas 10 is circulated in a closed loop inside the cabinet 1 between the cooling unit 9, the ventilation unit 8 and interior space 4. A control system 6, which may comprise a screen, control elements and a computer or a similar device, controls the operation of the cabinet, for example by controlling the ventilation unit 8 and the cooling unit 9. The cabinet 1 further comprises a drying unit 11. The drying unit 11 may be a separate unit or a single cooling and drying unit together with the cooling unit 9. The drying unit 11 dries the drying gas 10 in order to improve the drying capacity of the cabinet 1. The drying unit 11 and/or the ventilation unit 8 and/or the cooling unit 9 may comprise one or more filter 13 in order to filter contaminations from the drying gas 10.

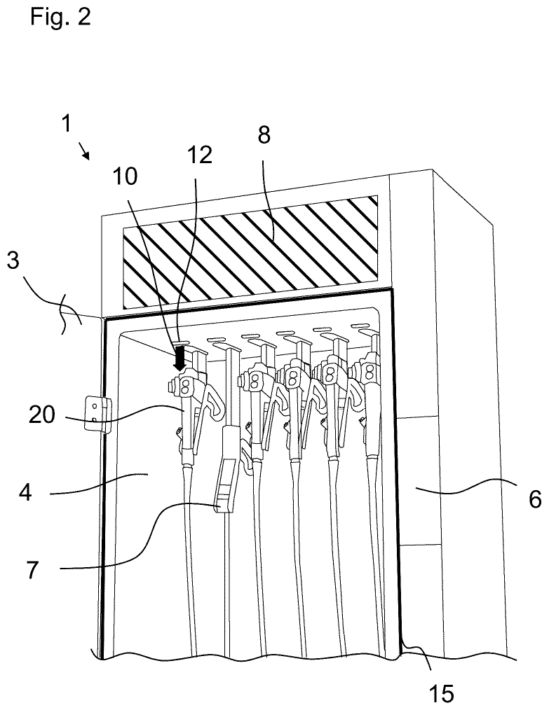

FIG. 2 shows a close-up perspective of the cabinet 1. In FIG. 2, several of the mounting devices 7, each hold a surgical instrument 20, in this case an endoscope. For simplicity sake, only one of the surgical instruments 20 and only one of the mounting devices 7 are provided with reference signs in FIGS. 1 and 2. As can be seen in FIG. 2, the cabinet 1 may comprise several vents 12 or nozzles. The vents 12 guide the drying gas 10 into the interior space 4 and may also guide the drying gas 10 back to the ventilation unit 8. In order to protect the interior channels of the surgical instruments 20 from recontamination, the drying gas 10 may also be guided as a drying gas steam through the interior channels of the surgical instruments 20. In order to guide the drying gas stream, the cabinet 1 may comprise a connection device, for example a connection tube, which is not shown in FIG. 2. An airtight seal 15 disposed around a periphery of the door opening seals the interior space 4 against the exterior when the doors 3 are closed.

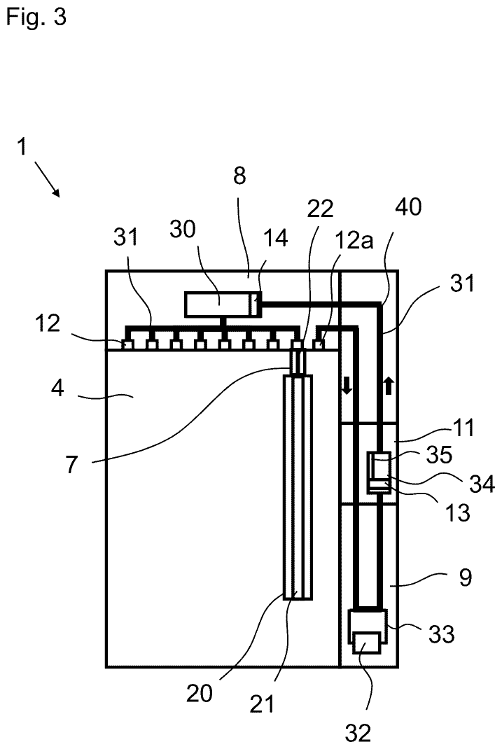

FIG. 3 shows a schematic simplified cross-section of another embodiment of the cabinet 1 for storing surgical instruments 20. In this embodiment, the ventilation unit 8 comprises one or more fans 30 and a ductwork comprising several ducts 31 to connect the fan 30 to the vents 12. For the sake of clarity, only one of the ducts 12 is marked with a reference sign. The surgical instrument 20, with is only schematically represented, comprises an interior channel 21, whose first end is connected to one of the ducts 12 via a connector 22 of the mounting device 7. The other end is open to the interior space 4, so that the drying gas 10 is guided through the inner channel 21 into the interior space 4. From the interior space 4, the drying gas 10 is guided in back into the ventilation unit 8 through an intake channel 12a. From there, the drying gas 10 is circulated through the cooling unit 9 and the drying unit 11. The cooling unit 9 comprises a compressor 32, which circulates a refrigerant through a coil 33 after the refrigerant is expanded and cooled. The coil 33 is at least partly disposed in the duct 31, where the fan 30 of the ventilation unit 8 pushes the drying gas across the coil 33. The drying unit 11 comprises a drying chamber 34. In the drying chamber 34, the humidity of the drying gas is reduced, for example with a silicate mineral 35 disposed within the drying chamber 34 in order to passively dry the drying gas. Alternatively, the drying chamber 34 may comprise active dehumidifiers, for example a condensation dehumidifier. Afterwards, the drying gas is cycled back to the ventilation unit 8. The drying unit 11 and the ventilation unit 8 may comprise filters 13, 14 which may filter out contaminations from the drying gas stream.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

1 cabinet
2 casing
3 door
4 interior space
5 sealing
6 control system
7 mounting device
8 ventilation unit
9 cooling unit
10 drying gas
11 drying unit
12 vents
12a intake channel
13 filter
14 filter
15 seal
20 surgical instrument
21 interior channel
22 connector
30 fan
31 duct
32 compressor
33 coil
34 drying chamber
35 silicate mineral
40 circulation system

What is claimed is:

1. A cabinet for storing surgical instruments the cabinet comprising:
a casing, wherein the casing encloses an interior space with at least one mounting device configured to hold a surgical instrument;
a ventilation unit configured to supply drying gas to the interior space; and
a cooling unit and a drying unit connected to the ventilation unit, wherein the drying unit is configured to reduce the humidity of the drying gas, and the cooling unit is configured to cool down the drying gas before it enters the interior space to a temperature below 15° C.

2. The cabinet according to claim 1, wherein the cooling unit is configured to cool down the drying gas before it enters the interior space to a temperature below 10° C.

3. The cabinet according to claim 1, wherein the cooling unit is configured to cool down the drying gas before it enters the interior space to a temperature in a range of 6° C. to 8° C.

4. The cabinet according to claim 1, wherein the interior space is thermally insulated and airtight against an exterior of the cabinet.

5. The cabinet according to claim 1, further comprising at least one door sealed against the casing with an airtight seal when in a closed state.

6. The cabinet according to claim 1, further comprising a closed off circulation system for the drying gas, wherein the closed off circulation system comprises the interior space, the ventilation unit and the cooling unit.

7. The cabinet according to claim 1, wherein the ventilation unit is configured to circulate the drying gas between the interior space and the cooling unit and the drying unit.

8. The cabinet according to claim 1, wherein one or more of the drying unit, ventilation unit and the cooling unit comprises a filter configured to continuously filter the drying gas.

9. The cabinet according to claim 1, wherein the ventilation unit is configured to supply the drying gas as a drying gas stream to at least one interior channel of a surgical instrument held in the mounting device.

10. The cabinet according to claim 1, wherein the cooling unit is configured to cool down the drying gas during a storage phase, which happens after a drying phase.

11. A method for operating a cabinet for storing surgical instruments, wherein the cabinet comprises a casing enclosing an interior space with at least one mounting device for holding a surgical instrument, a ventilation unit for supplying a drying gas to the interior space, a cooling unit connected to the ventilation unit, the method comprising:
the cooling unit cooling down the drying gas before it enters the interior space to a temperature below 15° C.

12. The method according to claim 11, wherein the cooling unit cools down the drying gas before it enters the interior space to a temperature below 10° C.

13. The method according to claim 11, wherein the cooling unit cools down the drying gas before it enters the interior space to a temperature in a range of 6° C. to 8° C.

14. The method according to claim 11, further comprising circulating the drying gas between the interior space and the cooling unit.

15. The method according to claim 11, further comprising continuously filtering the drying gas.

16. The method according to claim 11, further comprising the ventilation unit supplying the drying gas as a drying gas stream to at least one interior channel of the surgical instrument held in the mounting device.

17. The method according to claim 11, further comprising the cooling unit cooling down the drying gas during a storage phase, which happens after a drying phase.
